(19) United States
(12) Patent Application Publication
(10) Pub. No.: US 2010/0268119 A1
(43) Pub. Date: Oct. 21, 2010

(54) INTEGRATED FEEDBACK FOR IN-SITU SURGICAL DEVICE

(75) Inventor: Matthew M. MORRISON, Cordova, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., An Indiana Corporation, Warsaw, IN (US)

(21) Appl. No.: 12/423,849

(22) Filed: Apr. 15, 2009

(51) Int. Cl. A61B 5/103; A61B 17/84; A61F 5/00

(52) U.S. Cl. 600/587; 606/300; 606/86 A

(57) ABSTRACT

Apparatuses, systems, and methods including integrated feedback for in-situ surgical device contouring or bending are disclosed. One embodiment is a system including a bone anchor, a longitudinal member coupled with the bone anchor, an instrument adapted to bend the longitudinal member, a sensor configured to provide an output indicative of force exerted by the bone anchor on a bone coupled with the bone anchor when the instrument applies force to the longitudinal member, and an indicator configured to provide an indication when the output meets or exceeds a threshold. Another embodiment further includes a second sensor configured to provide a second output indicative of temperature of the longitudinal member and wherein the instrument is further configured to apply heat to the longitudinal member and the indicator is further configured to provide an indication when the second output meets or exceeds a second threshold.

### TECHNICAL FIELD

The present invention relates to apparatuses, systems, and methods including integrated feedback for in-situ contouring or bending of a surgical device.

### BACKGROUND

Stabilization and support of portions of the spinal column may be accomplished using one or more bone anchors that are coupled with bones at one or more locations along the spinal column, for example by implantation, and a rod that is engaged with the bone anchor(s) to provide stabilization and support to the spinal column. The rod may be initially provided in a substantially straight configuration, and subsequently bent or contoured in-situ to provide a desired positioning of the spinal column. Bending or contouring of such rods may be accomplished by imparting mechanical force on the rod either manually or using a variety of instruments. Such operations have presented a number of drawbacks and disadvantages including the possibility of degrading the interface between a bone anchor and the bone with which it is coupled. Thus, there is a need for additional contributions in this area of technology.

### SUMMARY

Apparatuses, systems, and methods including integrated feedback for in-situ surgical device contouring or bending are disclosed. One embodiment is a system including a bone anchor, a longitudinal member coupled with the bone anchor, an instrument adapted to bend the longitudinal member, a sensor configured to provide an output indicative of force exerted by the bone anchor on a bone coupled with the bone anchor when the instrument applies force to the longitudinal member, and an indicator configured to provide an indication when the output meets or exceeds a threshold. Other embodiments further include a second sensor configured to provide a second output indicative of temperature of the longitudinal member and wherein the instrument is further configured to apply heat to the longitudinal member and the indicator is further configured to provide an indication when the second output meets or exceeds a second threshold.

Another embodiment is a method including coupling a bone anchor to a bone, coupling a longitudinal member to the bone anchor, bending the longitudinal member when the bone anchor is coupled to the bone and the longitudinal member is coupled to the bone anchor, sensing a characteristic indicative of force exerted by the bone anchor on the bone during the bending, and providing an indication if the characteristic indicative of force meets or exceeds a threshold. Other embodiments further include applying heat to the longitudinal member, sensing a temperature of the longitudinal member, and providing an indication if the temperature of the longitudinal member meets or exceeds a temperature threshold.

Further embodiments, forms, features, aspects, benefits, objects and advantages of the present invention shall become apparent from the description and figures provided herewith.

### BRIEF DESCRIPTION OF THE FIGURES

FIG. 1 is a perspective view of an exemplary contouring system including integrated feedback for in-situ surgical device contouring in a first configuration.

FIG. 2 is a perspective view of the system of FIG. 1 in a second configuration.

FIG. 3 is a schematic block diagram of an exemplary contouring system including integrated feedback for in-situ surgical device contouring.

FIG. 4 is a flow diagram of an exemplary contouring method including integrated feedback for in-situ surgical device contouring.

### DETAILED DESCRIPTION

For the purposes of promoting an understanding of the principles of the invention, reference will now be made to the embodiments illustrated in the drawings and specific language will be used to describe the same. It will nevertheless be understood that no limitation on the scope of the invention is intended. Any alterations and further modifications in the illustrated devices and described methods and further applications of the principles of the invention as disclosed herein are contemplated as would occur to one skilled in the art to which the invention relates.

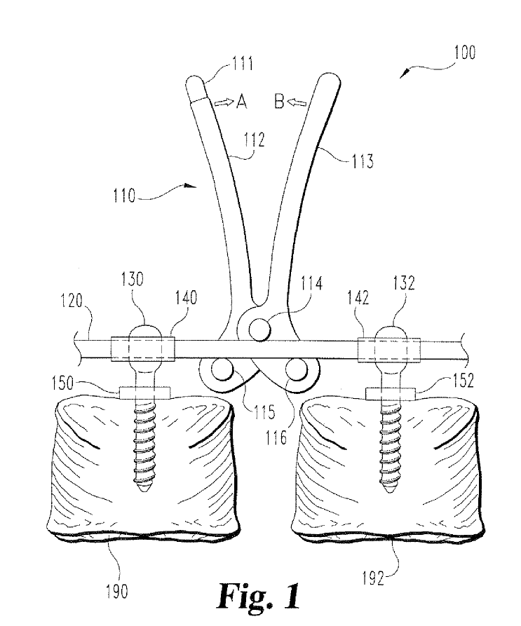

With reference to FIG. 1, there is illustrated a system 100 including integrated feedback for in-situ surgical device contouring. System 100 includes bone screws 130 and 132 which are implanted in vertebrae 190 and 192, respectively. In various embodiments bone screws 130 and 132 may be multiaxial bone screws or other types of bone anchors. System 100 further includes a rod 120 which is coupled to and extends between bone screws 130 and 132. In the illustrated embodiment rod 120 is a substantially cylindrical surgical rod. In other embodiments other longitudinal members such as plates or non-cylindrical rods may be used instead of, in addition to, or in combination with rod 120 and the characteristics and variations described in connection with rod 120 are also applicable to other longitudinal members. Various embodiments contemplate that longitudinal members such as rod 120 may be connected to patient anatomy in a variety of configurations, for example, along a portion of a length of the spinal column or along the length of substantially the entire spinal column, in a transverse relationship relative to the spinal column or a portion thereof, at angles relative to the length of the spinal column, in a substantially straight relationship relative to the spinal column or a portion thereof, in a curved relationship relative to the spinal column or a portion thereof, or in similar or other configurations with respect to other anatomical features such as long bones.

In the illustrated embodiment rod 120 is comprised of polyetheretherketone (PEEK). In other embodiments rod 120 may be comprised of additional or alternate thermoplastic polymers such as, for example, polycarbonate, polyketone, polyester, polyethylene, polyimide, polylactic acid, polypropylene, polystyrene, polysulfone, polyvinyl chloride, polyamide, poly(tetrafluoroethene), polyphthalamide, polybutylene and mixtures or combinations of thereof. In further embodiments rod 120 may be comprised of other materials which, when heated, facilitate bending of rod 120 to a desired configuration. In additional embodiments rod 120 may be formed from one or more metals or metal alloys, for example, surgical-grade titanium alloys or cobalt-chrome.

System 100 includes instrument 110 for bending or contouring rod 120. Instrument 110 includes a first lateral engaging member 115 positioned adjacent the distal end of handle member 112, a second lateral engaging member 116 positioned adjacent the distal end of handle member 113, and medial engaging member 114 positioned medially between the first and second lateral engaging members 115 and 116. In the illustrated embodiment, medial engaging member 114 includes a heating element and a temperature sensor. In other embodiments one or both of the heating element and the temperature sensor are provided in other locations on instrument 110, are provided separately from instrument 110 or are not present in system 100. Instrument 110 includes a pivotal interconnection between a first longitudinald handle member 112 and a second longitudinald handle member 113, which allows for relative pivotal movement about a pivot point or pivot axis. In the illustrated embodiment, the pivot point or pivot axis is located substantially at the center of medial engaging member 114. In additional embodiments the pivot point may be located in other positions. Further embodiments include compound pivot points or variable pivot points.

Instrument 110 further includes an indicator 111 positioned at the proximal end of handle member 112. Indicator 111 includes signal processing circuitry, for example, a microprocessor, or one or more application specific integrated circuits or ASICs. In other embodiments, signal processing circuitry may be provided independent of indicator 111 and may transmit information to indicator 111 via a communications link. In the illustrated embodiment indicator 111 receives information from one or more sensors via a wireless communications link. In other embodiments a physical communications link may be provided between indicator 111 and one or more sensors, for example, one or more electrical or optical interconnections. Indicator 111 is configured to provide an indication based upon evaluation of the output of one or more sensor as further described herein. In the illustrated embodiment, when the conditions of evaluation are satisfied, indicator 111 provides a visual indication by emitting visible light. Additional embodiments contemplate that indicator 111 could provide other visual indications, for example, alpha-numeric displays such as LED or liquid crystal displays, audible indications, or combinations of the foregoing and/or other indications.

The instrument 110 further includes a heating element which, in the illustrated embodiment, is integral to medial engagement member 114 and positioned to contact rod 120, but could also be provided independent from medial engagement member 114 and positioned in alternate locations. The heating element is operable to heat a portion of rod 120 positioned adjacent medial engaging member 114. The heating element may be configured to provide heat via convection heating, conduction heating, infrared heating or through other techniques. The heating element may utilize power from an internal or external power source to provide heat in a variety of manners including, for example, via a coil resistance heater, a metal oxide resistance heater, or a PTC (Positive Temperature Coefficient) heater. In one particular embodiment, the heating element comprises an infrared heating element. In other embodiments, the heating element comprises a band heater and/or a cartridge heater. In still other embodiments, the heating element directs hot air toward rod 120. Other suitable arrangements or configurations of the heating element are contemplated in addition to or in lieu of those described herein.

The heat applied to rod 120 by the heating element facilitates bending of rod 120 about the medial engaging member 114 to a desired configuration having a particular curvature or contour. In one exemplary embodiment rod 120 is formed of one or more heat deformable materials such as PEEK and the heating element heats the rod 120 until the thermoplastic polymer approaches or exceeds the glass transition temperature (T_{g}). As the thermoplastic polymer approaches or exceeds the glass transition temperature T_{g}, the material becomes more flexible. Once the rod 120 is heated in this manner, a surgeon may readily initiate bending of the rod 120 about the medial engaging member 114. For example, after a sufficient amount of heat is applied to rod 120 and rod 120 has achieved a sufficient degree of flexibility, a user may actuate the instrument 110 by pivoting the longitudinald handle members 112 and 113 toward one another, as indicated by arrows A and B, which in turn correspondingly pivots the first and second lateral engaging members 115 and 116 to apply force to rod 120 to initiate bending of rod 120 about the medial engaging member 114.

Instrument 110 includes a sensor which, in the illustrated embodiment, is provided in the medial engaging member 114 and is operable to provide an output indicative of the temperature of the rod. In other embodiments the sensor could be provided in other positions effective to provide an output indicative of the temperature of the rod. As further described below, the indication of the temperature of the rod evaluated against a condition and when evaluation conditions are satisfied, indicator 111 provides an indication as described above.

System 100 also includes sensor units 140, 142, 150 and 152. Sensor units 140 and 150 are configured to sense one or more characteristic(s) indicative of force exerted by the bone screw 130 on vertebra 190 when instrument 111 is used to bend rod 120 and to provide outputs indicative of that force. Sensor units 142 and 152 are configured to sense a characteristic indicative of force exerted by the bone screw 132 on vertebra 192 when instrument 111 is used to bend rod 120 and to provide outputs indicative of that force. Sensor units 140, 142, 150 and 152 may include one or more sensing elements such as strain sensors, metal foil strain gauges, piezoelectric sensors or other types of sensing elements. The sensing elements of system 100 may be arranged in an array to sense a characteristic indicative of force in a multiple directions. In other embodiments a single sensing element may be utilized. In other embodiments greater or fewer sensor units may be provided. In further embodiments sensor units may be positioned in other locations. Certain embodiments include a sensor array including one or more stress/strain sensor coupled with a bone anchor and one or more sensors coupled with a longitudinal member which are in communication with one another and/or with a processor operable process information from the sensor array to during in-situ application of force to the longitudinal member to identify and/or indicate when a desired threshold of force, stress and/or strain is achieved. In additional embodiments sensor units or sensing elements may be provided integral to bone screws 130 and 132 or other bone anchors. Output from sensor units 140, 142, 150 and 152 is provided to the signal processing circuitry of indicator 111 which evaluates whether one or more of the outputs received meets or exceeds a threshold. If the threshold is met or exceeded, indicator 111 provides an indication as described above.

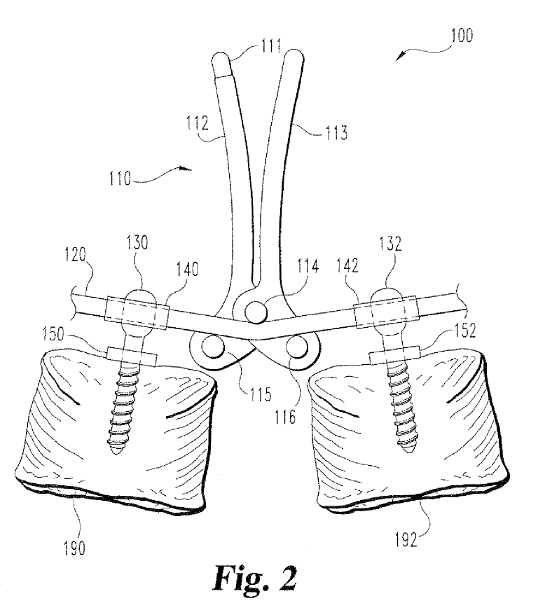

Bending of rod 120 begins from the configuration illustrated in FIG. 1 and proceeds to the configuration illustrated in FIG. 2. During bending, first and second handle members 112 and 113 are pivotally advanced toward one another generally in the direction indicated by directional arrows A and B. This movement correspondingly pivots or rotates the first and second lateral engaging members 115 and 116 away from one another to apply force to rod 120. Rod 120 is bent about the medial engaging member 114 to provide the rod 120 with a desired contour or curvature along its length. During this bending, output from sensors 140, 142, 150 and 152 is provided to the signal processing circuitry of indicator 111 which evaluates whether one or more of the outputs received meets or exceeds one or more thresholds and provides an indication if the threshold(s) is/are exceeded as described above.

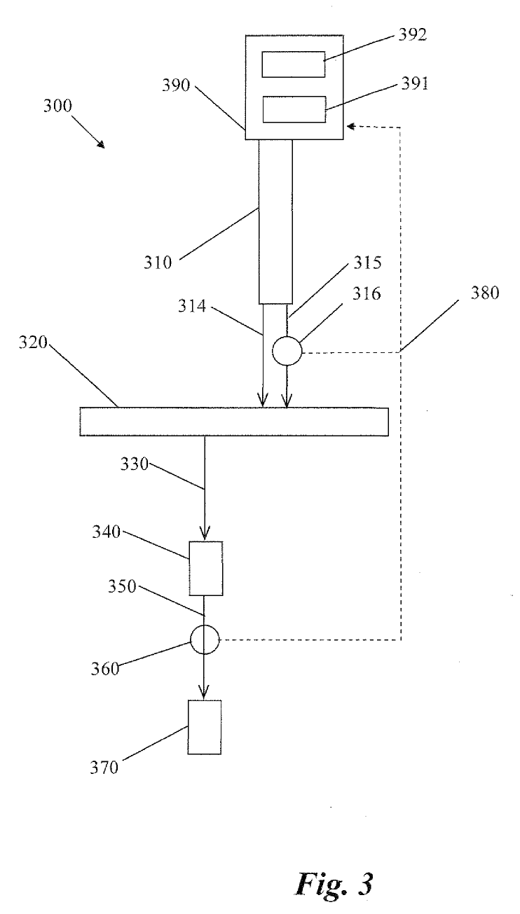

With reference to FIG. 3 there is illustrated a block diagram of an exemplary rod contouring system 300 including integrated feedback for in-situ surgical rod contouring. System 300 includes a bone anchor 340 which is coupled to a bone 370. In an embodiment bone anchor 340 is a multiaxial bone screw and bone 370 is a vertebra. In other embodiments bone anchor 340 may be another type of bone screw, a bone hook or another type of bone anchor. In certain embodiments the bone may be a different type of bone. System 300 also includes a rod 320 which is coupled to bone anchor 320, and an instrument which is engaged with rod 320. Other types of longitudinal members described herein may be used instead of, in addition to or in combination with rod 320. Rod 320 may be, for example, a PEEK rod or one of the other types of rods or other longitudinal members described herein. Instrument 310 may be, for example, the same as or similar to instrument 111 or another type of instrument adapted to bend a rod.

Instrument 310 is operated to apply a bending force 314 to rod 320. The application of bending force 314 results a in force 330 being exerted by rod 320 upon bone anchor 340. The exertion of force 330 upon bone anchor 340 results in a force 360 being exerted by bone anchor 340 upon bone 370. Sensor 360 is configured to sense a characteristic indicative of force 360. Sensor 360 provides an output indicative of force exerted by bone anchor 340 on a bone 370 to communications link 380 which provides the output to indicator 390. Communications link may be a wireless link or a physical link such as an electrical or optical link. Indicator 390 includes processing circuitry 391 which is configured to evaluate whether the output of sensor 360 meets or exceeds a threshold. The threshold is determined based upon a maximum force desired to be exerted by bone anchor 340 on bone 370. When processing circuitry 391 determines that the threshold is met or exceeded it triggers indicator output 392 to provide an indication that the characteristic indicative of force meets or exceeds a threshold.

Instrument 310 is may also be operated to apply heat 315 to a portion of rod 320. Sensor 316 is configured to sense the temperature of rod 320 at or near the location of heat application. Sensor 360 provides an output indicative of the sensed temperature to communications link 380 which provides the output to indicator 390. In other embodiments a separate communications link may be used. Indicator 390 includes processing circuitry 391 which is configured to test whether the output of sensor 316 meets or exceeds a threshold. The threshold is determined based upon the glass transition temperature (T_{g}) of the rod. When processing circuitry 391 determines that the threshold is met or exceeded it triggers indicator output 392 to provide an indication that the temperature meets or exceeds a threshold.

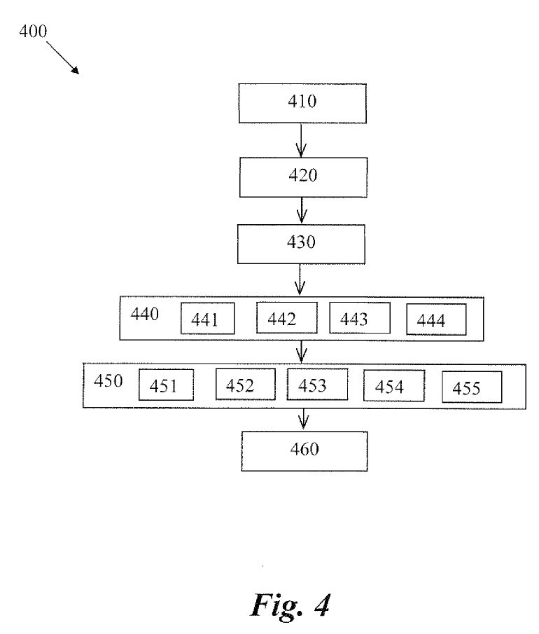

With reference to FIG. 4 there is illustrated a flow diagram of an exemplary contouring process 400 including integrated feedback for in-situ surgical device contouring. Process 400 begins at operation 410 where one or more bone anchors are coupled with one or more bones. In certain embodiments the bone anchors are multiaxial bone screws. In other embodiments the bone anchors are other types of bone screws. In further embodiments, the bone anchors are other types of osteo-implants. In additional embodiments the bone anchors are bone hooks. In certain embodiments the bones are vertebrae. From operation 410 process 400 proceeds to operation 420.

At operation 420 a rod is coupled with one or more of the bone anchors described above. In certain embodiments the rod is comprised of polyetheretherketone (PEEK). In other embodiments the rod is comprised of additional or alternate thermoplastic polymers such as, for example, polycarbonate, polyketone, polyester, polyethylene, polyimide, polylactic acid, polypropylene, polystyrene, polysulfone, polyvinyl chloride, polyamide, poly(tetrafluoroethene), polyphthalamide, polybutylene and mixtures or combinations of thereof. In further embodiments the rod is comprised of other materials which, when heated, facilitate bending to a desired configuration having a particular curvature or contour. In additional embodiments the rod is comprised of one or more metals or metal alloys, for example, surgical-grade titanium alloys or cobalt-chrome. In certain embodiments the rod has a substantially circular cross sectional shape. In other embodiments the rod has other circular cross sectional shapes, for example, oval, ellipsoid, tapered, conic-sectional, I-shaped, H-shaped, rectangular, square or other polygonal cross sectional shapes. In certain embodiments other longitudinal members such as plates or non-cylindrical rods may be used instead of, in addition to, or in combination the rod at operation 420. From operation 420 process 400 proceeds to operation 430.

At operation 430 at least one instrument is engaged with the rod described above. In certain embodiments the instrument is instrument 110 described hereinabove. In further embodiments the instrument is another type of instrument adapted to bend a rod. In certain embodiments the instrument engages the rod in three contact locations. In alternate embodiments the instrument engages the rod in two contact locations. In other embodiments the instrument engages the rod in a different number of contact locations. In certain embodiments one instrument is engaged with the rod. In other embodiments, two or more instruments are engaged with the rod. From operation 430 process 400 proceeds to operation group 440.

Operation group 440 includes operations 441, 442, 443, and 444 which may be performed in parallel, in series, in a combination of parallel and series, and may be temporally coextensive, temporally overlapping, temporally contiguous, or temporally separated. At operation 441 heat is applied to the rod. In certain embodiments heat is applied to the rod using an instrument engaged with the rod. In alternate embodiments heat is applied to the rod using a heat source independent of the instrument engaged with the rod.

At operation 442 the temperature of the rod is sensed. In certain embodiments the temperature of the rod is sensed using a temperature sensor provided on the instrument engaged with the rod. In other embodiments the temperature of the rod is sensed using a temperature sensor independent from the instrument engaged with the rod. The temperature sensor outputs information indicative of the temperature of the rod which is transmitted via a communication link to processing circuitry. In certain embodiments the communications link is a wireless communications link. In other embodiments the communications link is a physical communications and may include one or more electrical or optical interconnections.

At operation 443 the processing circuitry evaluates the information received from the sensor against one or more predetermined criteria. In certain embodiments the processing circuitry includes a microprocessor and associated memory. In certain embodiments the processing circuitry includes an application specific integrated circuit or ASIC. In certain embodiments the processing circuitry tests whether the information indicative of the temperature of the rod meets or exceeds a threshold. In some embodiments the threshold in selected based upon the glass transition temperature (T_{g}) of the rod. In certain embodiments the threshold may be set to T_{g}. In some embodiments the threshold may be set to T_{g}. plus or minus 2%, 5% or 10% or another percentage. In some embodiments the threshold may be set to T_{g} plus or minus 5° F., 10° F, 15° F, 20° F. or another temperature differential of T_{g}. If and when the predetermined criteria is satisfied the processing circuitry sends a signal to activate an indicator.

At operation 444 an indicator provides an indication if the temperature of the rod meets or exceeds the threshold. In certain embodiments the indicator provides a visual indication by emitting visible light. Additional embodiments contemplate that the indicator could provide other visual indications, for example, alpha-numeric displays such as LED or liquid crystal displays, audible indications, or combinations of the foregoing and other indications.

From operation group 440 process proceeds to operation group 450. Certain embodiments contemplate that one or more operations in operation group 440 may be omitted. For example, operation 441 alone may be performed, or all operations in operation group 440 may be omitted and operation 400 may proceed from operation 430 to operation group 450.

Operation group 450 includes operations 451, 452, 453, 454 and 455 which may be performed in parallel, in series, in a combination of parallel and series, and may be temporally coextensive, temporally overlapping, temporally contiguous or temporally separated. At operation 451 one or more sensors may be attached to the bone anchor. Certain embodiments may include sensors which are integral to the bone anchor or which are pre-attached thereto and in such embodiments operation 451 may be omitted. At operation 452 the rod is bent or contoured by applying force to the rod using the instrument engaged with the rod.

At operation 453 a characteristic indicative of force exerted by the bone anchor on the bone is sensed during the bending. One or more sensors may provide an output indicative of force exerted by the bone anchor rod which is transmitted via a communications link to processing circuitry. In certain embodiments the communications link is the same link described above in connection with operation 442 or a portion thereof. In other embodiments the communications link is a separate communications link.

At operation 454 the processing circuitry evaluates the information received from the sensor against one or more predetermined criteria. In certain embodiments the processing circuitry is the same processing circuitry described above in connection with operation 453. In other embodiments separate processing circuitry is used. In certain embodiments the processing circuitry tests whether the output of one or more sensors meets or exceeds a threshold based upon a maximum force that bone anchor should be permitted to exert on the bone with which it is coupled.

At operation 455 an indicator provides an indication if the output indicative of force meets or exceeds a threshold. In certain embodiments the indicator provides a visual indication by emitting visible light. Additional embodiments contemplate that the indicator could provide other visual indications, for example, alpha-numeric displays such as LED or liquid crystal displays, audible indications, or combinations of the foregoing and other indications. In some embodiments the indicator is the same indicator described above in connection with operation 444. In other embodiments a separate indicator is used.

From operation group 450 process 400 proceeds to operation 460. At operation 460 process 400 may be complete or, alternatively, process 400 may proceed to any of the previously described operations or operation groups and proceed as described above.

Any theory, mechanism of operation, proof, or finding stated herein is meant to further enhance understanding of the present invention, and is not intended to make the present invention in any way dependent upon such theory, mechanism of operation, proof or finding. It should be understood that while the use of the word preferable, preferably or preferred in the description above indicates that the feature so described may be more desirable, it nonetheless may not be necessary, and embodiments lacking the same may be contemplated as within the scope of the application, that scope being defined by the claims that follow. In reading the claims, it is intended that when words such as “a”, “an”, “at least one”, and “at least a portion” are used, there is no intention to limit the claim to only one item unless specifically stated to the contrary in the claim. Further, when the language “at least a portion” and/or “a portion” is used, the item may include a portion and/or the entire item unless specifically stated to the contrary.

While the application has been illustrated and described in detail in the drawings and foregoing description, the same is to be considered as illustrative and not restrictive in character, it being understood that only the selected embodiments have been shown and described and that all changes, modifications and equivalents that come within the spirit of the invention as defined herein or by any of the following claims are desired to be protected.

What is claimed is:

1. A system comprising:
a bone anchor;
a longitudinal member coupled with the bone anchor;
an instrument adapted to bend the longitudinal member;
a sensor configured to provide an output indicative of force exerted by the bone anchor on a bone coupled with the bone anchor when the instrument applies force to the longitudinal member; and
an indicator configured to provide an indication when the output meets or exceeds a threshold.

2. A system according to claim 1 wherein the sensor is a strain sensor configured to sense a strain of the bone anchor.

3. A system according to claim 1 wherein the sensor is integral to the bone anchor.

4. A system according to claim 1 wherein the indicator is coupled with the instrument.

5. A system according to claim 1 further comprising a second sensor configured to provide a second output indicative of temperature of the longitudinal member and wherein the instrument is further configured to apply heat to the longitudinal member and the indicator is further configured to provide an indication when the second output meets or exceeds a second threshold.

6. A system according to claim 5 wherein the longitudinal member includes a thermoplastic having a glass transition temperature and the threshold is selected based upon the glass transition temperature of the thermoplastic.

7. A system according to claim 1 further comprising:
a second bone anchor coupled with the longitudinal member; and
a second sensor configured to provide a second output indicative of force exerted by the second bone anchor on a second bone coupled with the second bone anchor when the instrument applies force to the longitudinal member; wherein
the indicator is further configured to provide an indication when the second output meets or exceeds a second threshold.

8. A system according to claim 1 wherein the bone anchor is a multiaxial bone screw.

9. A system according to claim 1 wherein the indication includes visible light.

10. A system according to claim 1 wherein the longitudinal member is a substantially cylindrical rod.

11. A system according to claim 7 wherein the first bone and the second bone are vertebrae.

11. A method comprising:
coupling a bone anchor to a bone;
coupling a longitudinal member to the bone anchor;
bending the longitudinal member when the bone anchor is coupled to the bone and the longitudinal member is coupled to the bone anchor;
sensing a characteristic indicative of force exerted by the bone anchor on the bone during the bending; and
providing an indication if the characteristic indicative of force meets or exceeds a threshold.

12. A method according to claim 11 wherein the characteristic indicative of force exerted by the bone anchor on the bone includes a strain of the bone anchor.

13. A method according to claim 11 wherein the providing an indication includes providing a visible indication.

14. A method according to claim 11 further comprising heating the longitudinal member prior to the bending.

15. A method according to claim 11 further comprising attaching a sensor to the bone anchor before the sensing.

16. A method according to claim 15 further comprising removing the sensor after the bending.

17. A method according to claim 11 further comprising:
applying heat to the longitudinal member;
sensing a temperature of the longitudinal member; and
providing an indication if the temperature of the longitudinal member meets or exceeds a temperature threshold.

18. A method according to claim 17 wherein the temperature threshold is selected based upon a glass transition temperature of the longitudinal member.

19. A method according to claim 11 further comprising:
coupling a second bone anchor to a second bone before the bending;
coupling the longitudinal member to the second bone anchor before the bending;
sensing a second characteristic indicative of force exerted by the second bone anchor on the second bone during the bending; and
providing an indication if the second characteristic indicative of force meets or exceeds a second threshold.

20. A method according to claim 11 wherein the longitudinal member is a surgical rod and the bone is a vertebra.
